(19) United States
(12) Patent Application Publication
(10) Pub. No.: US 2012/0167892 A1
(43) Pub. Date: Jul. 5, 2012

(54) PATIENT INTERFACE DEVICE INCLUDING A MECHANISM FOR MANIPULATING THE POSITION OF AN INTERNAL COMPONENT THEREOF

(75) Inventor: Jerome Matula, JR., Apollo, PA (US)

(73) Assignee: KONINKLIJKE PHILIPS ELECTRONICS N.V., EINDHOVEN (NL)

(21) Appl. No.: 13/395,519

(22) PCT Filed: Aug. 17, 2010
(86) PCT No.: PCT/IB2010/053713

§ 371 Date: Mar. 12, 2012

Related U.S. Application Data
(60) Provisional application No. 61/241,512, filed on Sep. 11, 2009.

(51) Int. Cl. A61M 16/06; A61M 15/08

(52) U.S. Cl. 128/206.21

(57) ABSTRACT

A patient interface device includes a mask having a shell defining a chamber. A component, such as a nasal cannula coupled to a sensor, is structured to be selectively positioned within the chamber. A manipulation mechanism for selectively manipulating the position of the component within the chamber from an exterior of the mask when the mask is placed against the patient's face. The manipulation mechanism may include an attachment element, such as a magnet, attached to the component and structured to be selectively positioned on the interior surface of the shell, and a manipulation element, such as a magnet, structured to be selectively positioned on the exterior surface of the shell and coupled to the attachment element through the shell, wherein movement of the manipulation element causes movement of the attachment element and the component.

### CROSS-REFERENCE TO RELATED APPLICATIONS

This patent application claims the priority benefit under 35 U.S.C. §119(e) of U.S. provisional application No. 61/241,512 filed on Sep. 11, 2009, the contents of which are herein incorporated by reference.

### BACKGROUND OF THE INVENTION

1. Field of the Invention

The present invention relates to patient interface devices for transporting a gas to and/or from an airway of a user, and in particular, to a mechanism for manipulating the position of an internal component of the patient interface device, such as a nasal cannula and associated tubing coupled to a sensor, from the exterior of the patient interface device.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device, which is typically a nasal mask that covers the nose, a nasal/oral mask that covers the nose and mouth, or full face mask that covers the patient face, on the face of a patient. The patient interface device interfaces the ventilator or pressure support device with the airway of the patient, so that a flow of breathing gas can be delivered from the pressure/flow generating device to the airway of the patient. It is known to maintain such masks on the face of a wearer by a headgear having one or more straps adapted to fit over/around the patient's head.

During use, particularly in cases of non-invasive ventilation, it is often necessary for a caregiver to position a component within the mask. One example of such a component includes an apparatus, such as a nasal cannula and associated tubing, that collects and communicates gas (e.g., expired gas) from within the mask to a sensor, such as an oxygen sensor, a carbon dioxide sensor or a pressure sensor, for measurement and/or analysis of constituents of and/or parameters relating to the gas. It is also know to position a nasal-gastric tubing within a mask for providing an item, such as food to the patient.

In current systems, the caregiver must remove the mask (i.e., break the seal with the patient's face), position the component as desired, and reposition the mask on the patient's face. These steps must be repeated when the use of the internal component (e.g., nasal cannula, etc.) is complete. This process is inconvenient to both the caregiver and the patient and disturbs the seal that has been previously established between the mask and the patient's face.

Thus, there is a need for a solution that enables the position of an internal component of a patient interface device to be manipulated from the exterior of the patient interface device without the need to remove the patient interface device and/or disturb a previously established seal between the patient interface device and the patient's face.

### SUMMARY OF THE INVENTION

In one exemplary embodiment, a patient interface device is provided that includes a mask having a shell, wherein the shell has an interior surface and an exterior surface and the shell defines a chamber when the mask is placed against a face of a patient, a component structured to be selectively positioned within the chamber defined by the shell, and a manipulation mechanism for selectively manipulating the position of the component within the chamber from the exterior of the mask when the mask is placed against the face of the patient.

In one particular exemplary embodiment, the manipulation mechanism includes an attachment element attached to the component and structured to be selectively positioned on the interior surface of the shell, and a manipulation element structured to be selectively positioned on the exterior surface of the shell and coupled to the attachment element through the shell, wherein movement of the manipulation element causes movement of the attachment element and the component. The present invention contemplates that the attachment element includes at least one first magnet, the manipulation element includes at least one second magnet, and the attachment element is coupled to the manipulation element by a magnetic attraction between the at least one first magnet and the at least one second magnet through the shell. The first magnet may be two first disk shaped magnets and the second magnet may be two second disk shaped magnets. Also, the manipulation element may include a handle element coupled to the at least one second magnet for selectively moving the manipulation element on the exterior surface of the shell.

In a further embodiment, the manipulation mechanism includes a second attachment element attached to the component and structured to be selectively positioned on the interior surface of the shell, and a second manipulation element structured to be selectively positioned on the exterior surface of the shell and coupled to the second attachment element through the shell, wherein movement of the second manipulation element causes movement of the second attachment element and the component.

The component that is movable may be a fluid communicating device, such as a fluid communicating device that includes a nasal cannula coupled to at least one tubing portion. The patient interface device may also include an interior port provided on the interior surface, and an exterior port provided on the exterior surface, wherein the interior port is in fluid communication with the exterior port, and wherein the at least one tubing portion is fluidly coupled to the interior port.

In an alternative embodiment, the fluid communicating device includes a tubing portion coupled to a contoured seal element, wherein the contoured seal element is structured to be positioned between a compliant rim of the mask and the face of the patient. The contoured seal element has a flat base for engaging the face of the patient and a contoured top surface for engaging the rim, wherein the contoured top surface has a concave central portion and first and second end portions that taper to meet the flat base.

In another embodiment, a method is providing for adjusting the position of a component of a patient interface device having a mask having a shell having an interior surface and an exterior surface. The method includes positioning an attachment element on the interior surface, the attachment element being attached to the component, positioning a manipulation element on the exterior surface of the shell and coupling the manipulation element to the attachment element through the shell, placing the mask against the face of a patient, the shell defining a chamber when the mask is placed against the face of the patient, and moving the manipulation element on the exterior surface, wherein the movement of the manipulation element causes movement of the attachment element and movement of the component within the chamber. The method may further include positioning a second attachment element on the interior surface, the second attachment element being attached to the component, positioning a second manipulation element on the exterior surface of the shell and coupling the second manipulation element to the second attachment element through the shell, and moving the second manipulation element on the exterior surface, wherein the movement of the second manipulation element causes movement of the second attachment element and movement of the component within the chamber.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various FIGS. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is front perspective view of a patient interface device according to one particular embodiment of the present invention;

FIG. 2 is partial rear perspective view of the patient interface device of FIG. 1;

FIGS. 3A, 3B and 3C are front elevational, side elevational and top plan views, respectively, of the magnetic attachment element forming a part of the patient interface device of FIG. 1;

FIG. 3D is a side elevational view showing the attachment of the magnetic attachment element forming a part of the patient interface device of FIG. 1 to a tube portion of the patient interface device of FIG. 1;

FIGS. 4A, 4B and 4C are front elevational, side elevational and top plan views, respectively, of the magnetic manipulation element forming a part of the patient interface device of FIG. 1;

FIG. 5 is a partial side view showing an attachment/manipulation assembly of the patient interface device of FIG. 1 in an engaged/coupled condition;

FIG. 6 is front perspective view of the patient interface device according to one particular embodiment of the present invention showing a first attachment/manipulation assembly in an engaged/coupled condition and a second attachment/manipulation assembly prior to being engaged/coupled;

FIG. 7 is an perspective view of a fluid communicating device employing a contoured seal element according to an alternative embodiment of the present invention;

FIG. 8 is a side, cross-sectional view showing a mask employing the fluid communicating device contoured seal element while in use; and

FIG. 9 is a perspective view of an exemplary embodiment of the contoured seal element.

### DETAILED DESCRIPTION OF EXEMPLARY EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein. As employed, herein, the statement that two or more parts or components are “coupled” together shall mean that the parts are joined or operate together either directly or through one or more intermediate parts or components. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

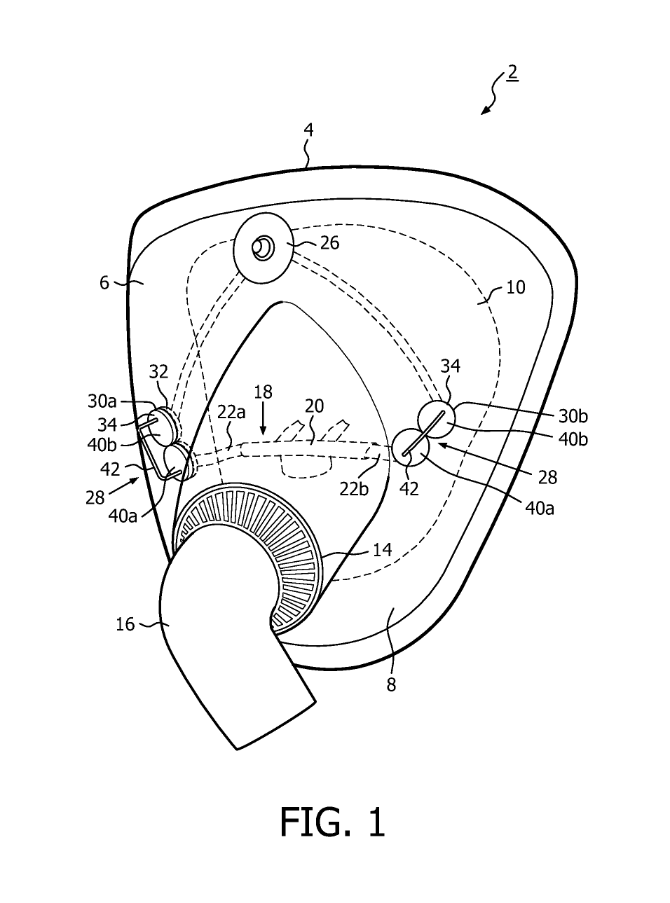

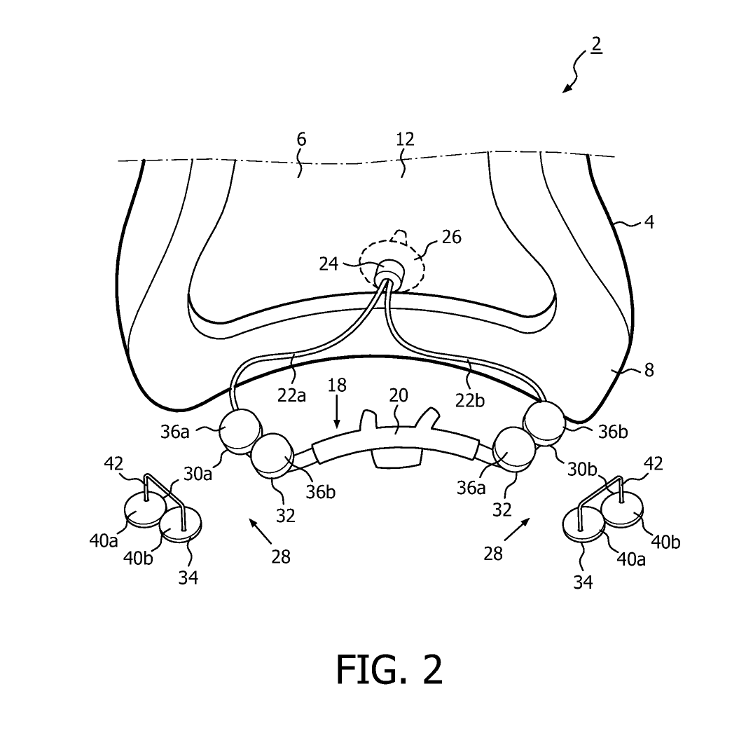

FIGS. 1 and 2 depict a patient interface device 2 according to one particular embodiment of the present invention. Patient interface device 2 includes face mask 4 having a shell 6 defining a chamber therein bounded by flexible, compliant peripheral rim 8, made of, for example, an elastomer such as plastic, rubber, silicone, vinyl or foam, for sealing against the face of a patient.

Although face mask shown 4 shown in the particular embodiment is a nasal/oral mask, it should be understood that that is meant to be exemplary only and that other types of masks, such as, without limitation, nasal masks and full face masks, may also be employed in the present invention.

Shell 6 includes exterior surface 10 and interior surface 12, and is typically made of a transparent or translucent material such as a transparent or translucent plastic, rubber or silicone. Shell 6 also defines opening 14 to which there is attached fluid coupling device 16, such as a swivel conduit, for carrying a fluid, such as a breathing gas, between the chamber within patient interface device 2 and an external gas source (not shown), such as a blower or other suitable device.

Patient interface device 2 further includes fluid communicating device 18 in the form of nasal cannula 20 coupled to first and second tubing portions 22a and 22b. Fluid communicating device 18 is structured to be disposed on the interior side of shell 6 within the chamber defined by shell 6. Interior port 24 is provided on interior surface 12 of shell 6. First and second tubing portions 22a and 22b are coupled to interior port 24. Interior port 24 is fluidly coupled to exterior port 26 provided on exterior surface 10 of shell 6. Exterior port 26 is structured to be coupled, e.g., via tubing (not shown), to a sample cell and associated sensor (not shown) to enable gas from within patient interface device 2 to be transported to the sample cell and sensor for measurement and/or analysis of constituents of and/or parameters relating to the gas.

According to an aspect of the present invention, patient interface device 2 includes a manipulation mechanism 28 for selectively manipulating the position of fluid communicating device 18 within the within the chamber defined by shell 6 from the exterior of patient interface device 2 (when the patent interface device is worn by the patient). In the particular embodiment of patient interface device 2 shown in FIGS. 1 and 2, manipulation mechanism 28 includes first attachment/manipulation assembly 30a and second attachment/manipulation assembly 30b. Each attachment/manipulation assembly 30a, 30b includes a magnetic attachment element 32 and a magnetic manipulation element 34 which may be selectively coupled to one another as described below.

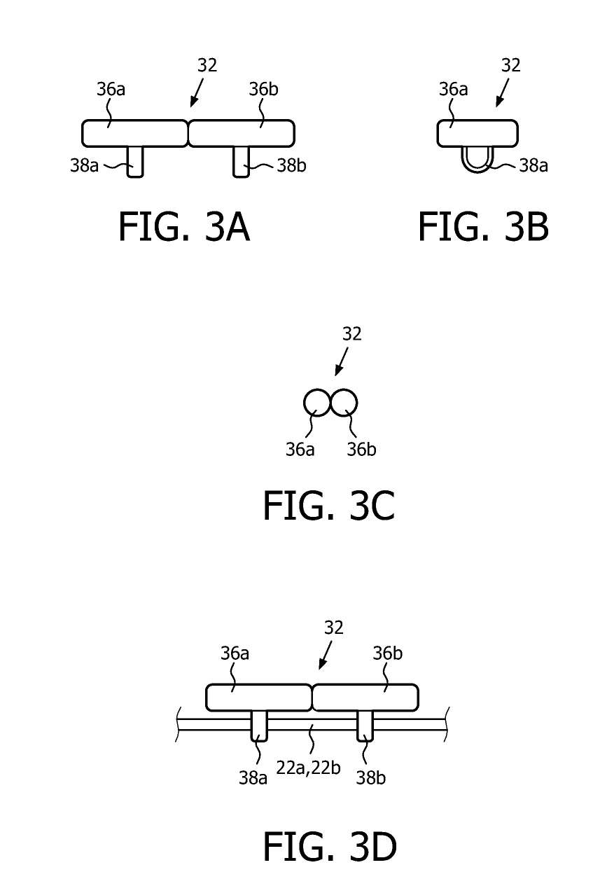

FIGS. 3A, 3B, and 3C are front elevational, side elevational and top plan views, respectively, of magnetic attachment element 32 according to one particular embodiment. As seen in FIGS. 3A, 3B, and 3C, magnetic attachment element 32 includes first and second disk shaped magnets 36a and 36b attached and positioned adjacent to one another. In addition, magnetic attachment element 32 includes loop portion 38a on a bottom surface of disk shaped magnet 36a and loop portion 38b on a bottom surface of disk shaped magnet 36b. As seen in FIG. 3D, and as described elsewhere herein, loop portions 38a and 38b are structured to receive therethrough either tubing portion 22a or tubing portion 22b and to hold tubing portion 22a or tubing portion 22b by a friction fit. This configuration thus allows magnetic attachment element 32 to be attached to fluid communicating device 18, the purpose of which is described below.

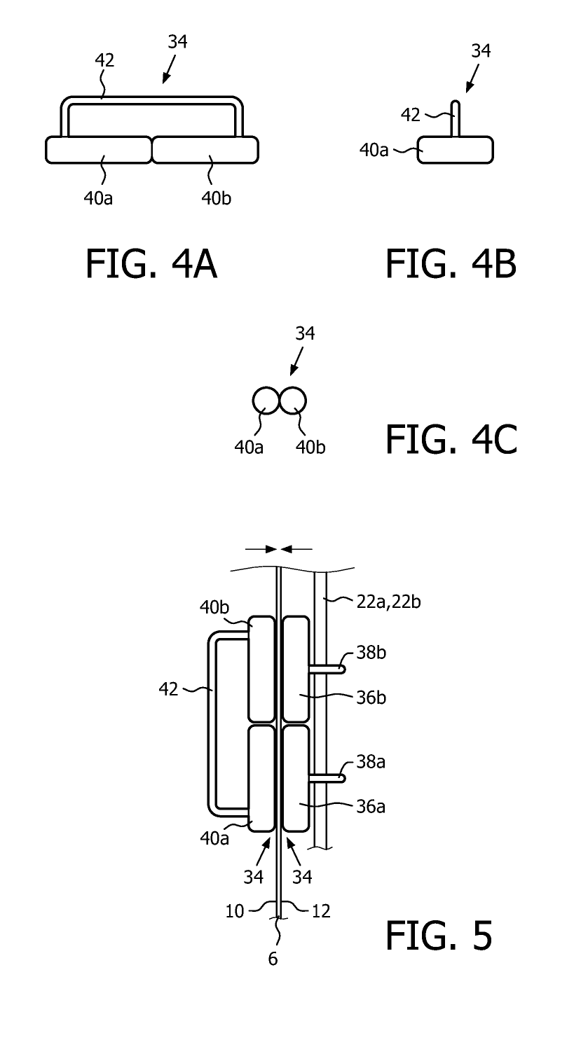

FIGS. 4A, 4B, and 4C are front elevational, side elevational and top plan views, respectively, of magnetic manipulation element 34 according to one particular embodiment. As seen in FIGS. 4A, 4B, and 4C, magnetic manipulation element 34 includes first and second disk shaped magnets 40a and 40b attached and positioned adjacent to one another. In addition, magnetic manipulation element 34 includes handle element 42 extending upwardly from the top surface thereof. More specifically, the first end of handle element 42 is attached to the top surface of disk shaped magnet 40a and the opposite end of handle element 42 is attached to the top surface of disk shaped magnet 40b. Handle element 42 provides a structure that can be gripped to enable selective movement of magnetic manipulation element 34 as described below.

In operation, fluid communicating device 18 is attached to shell 6 by placing the top surface of disk shaped magnets 36a and 36b of each magnetic attachment element 32 against interior surface 12 of shell 6 as shown in FIG. 5. In such a configuration, one pole (North or South) of each disk shaped magnet 36a and 36b will engage interior surface 12 of shell 6. A magnetic manipulation element 34 is then placed on exterior surface 10 of shell 6 opposite each magnetic attachment element 32 in an orientation wherein disk shaped magnets 36a and 36b are aligned with disk shaped magnets 40a and 40b as shown in FIG. 5. In addition, magnetic manipulation elements 34 are structured so that the pole (North or South) of each disk shaped magnet 40a and 40b that engages exterior surface 10 of shell 6 is opposite of the pole of each corresponding disk shaped magnet 36a and 36b that engages interior surface 12 of shell 6. As a result, magnetic attachment elements 32 and magnetic manipulation elements 34 will be magnetically attracted to one another such that they are coupled together and held in place on shell 6.

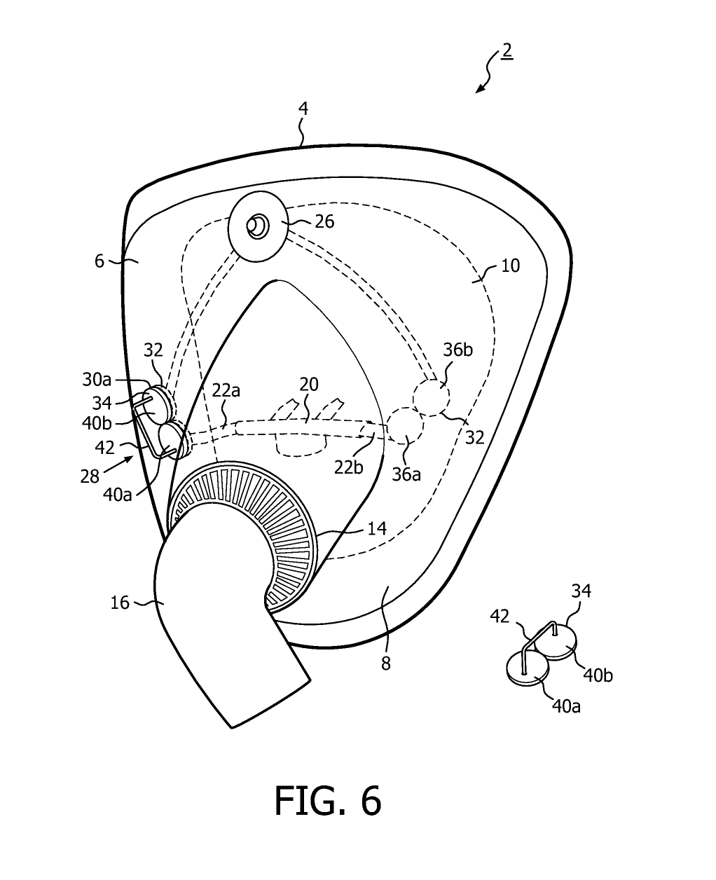

Because fluid communicating device 18 is attached to each magnetic attachment element 32, it will also be held in place within the chamber defined by shell 6. FIG. 6 is an isometric view of patient interface device 2 showing first attachment/manipulation assembly 30a in an engaged/coupled condition and second attachment/manipulation assembly 30b prior to being engaged/coupled as described above. When first attachment/manipulation assembly 30a and second attachment/manipulation assembly 30b are both in an engaged/coupled condition, patient interface device 2 may then be placed on the face of the patient.

In order to selectively position fluid communicating device 18 within the chamber defined by shell 6 (for example, to position nasal cannula 20 so that gas may be collected and analyzed), a caregiver or patient may simply grip handle element 42 of each magnetic manipulation element 34 and move it as desired (for example, so that the prongs of nasal cannula 20 enter the patient's nostrils). The magnetic attraction between each magnetic manipulation element 34 and each corresponding magnetic attachment element 32 will cause the corresponding magnetic attachment element 32 to be similarly moved (slide) along interior surface 12 of shell 6. As a result, attached fluid communicating device 18 will also be moved within the chamber defined by shell 6. Magnetic manipulation elements 34 may be independently moved in this manner until fluid communicating device 18 is positioned as desired. As will be appreciated, such movement may be affected entirely from the exterior of face mask 4 without the need to remove patient interface device 2 from the face of the patient.

The particular embodiment of patient interface device 2 shown in FIGS. 1 and 2 that employs manipulation mechanism 28 having two attachment/manipulation assemblies, first attachment/manipulation assembly 30a and second attachment/manipulation assembly 30b, is particularly advantageous in that it provides not only a single point locating ability, but also the ability to rotate the fluid communicating device 18 to fine tune the positioning thereof. This features provides the ability to rotate the nasal cannula 20 and first and second tubing portions 22a and 22b relative to the patient's nose (i.e., inward towards the nostrils, or away from the nostrils).

As described above, fluid communicating device 18 requires a mask, like mask 4, having an interior port 24 and an exterior port 26 to enable gas to be communicated from within the mask to a sensor outside of the mask. Most current masks do not have such ports, and thus fluid communication device 18 coupled to manipulation mechanism 28 shown in FIGS. 1-6 cannot be effectively used therewith. It would be advantageous, however, to be able to utilize the principles of manipulation mechanism 28 on masks that do not have ports 24, 26.

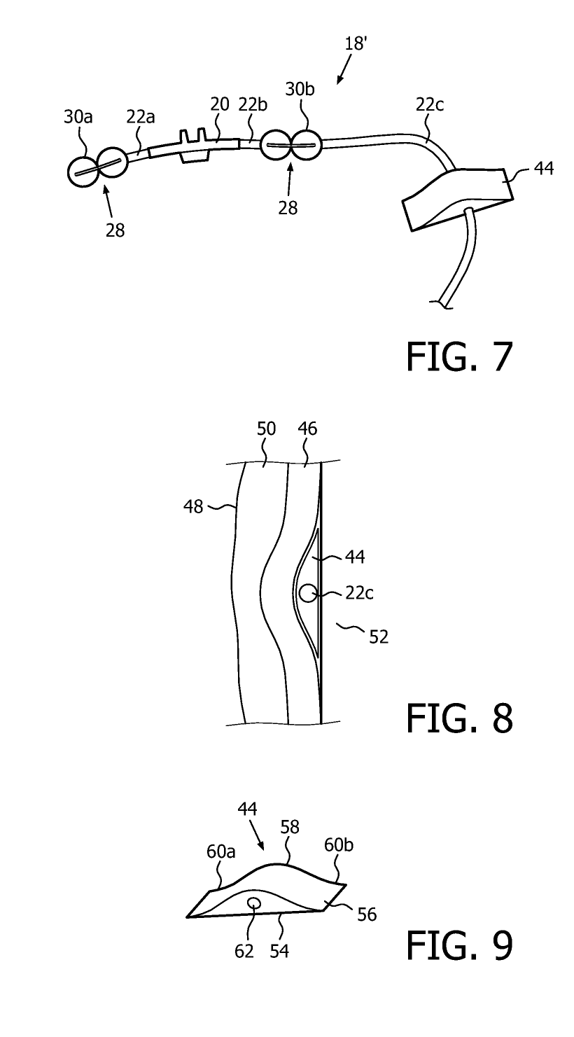

FIG. 7 is an isometric view of fluid communicating device 18′ according to an alternative embodiment of the present invention that enables the principles of manipulation mechanism 28 to be used on masks that do not have ports 24, 26. As seen in FIG. 7, fluid communicating device 18′ is coupled to manipulation mechanism 28 having two attachment/manipulation assemblies, first attachment/manipulation assembly 30a and second attachment/manipulation assembly 30b, as described elsewhere herein. Fluid communicating device 18′ coupled to manipulation mechanism 28, like fluid communication device 18 coupled to manipulation mechanism 28 described above (FIGS. 1-6), allows for the selective positioning of fluid communicating device 18′ within a chamber defined by a mask from the outside of the mask without breaking a seal between the mask and the patient's face. However, as described below, fluid communicating device 18′ coupled to manipulation mechanism 28 is structured so that it may be used with masks not having the ports just described.

As seen in FIG. 7, fluid communicating device 18′ includes nasal cannula 20 coupled to first and second tubing portions 22a and 22b. Fluid communicating device 18′ also includes third tubing portion 22c that is inserted through contoured seal element 44. As described in greater detail below, and as shown in FIG. 8, contoured seal element 44 is structured to be positioned between (i) flexible, compliant peripheral rim 46 of mask 48 having shell 50 and (ii) face 52 of a patient, while enabling nasal cannula 20 and first and second tubing portions 22a and 22b to be positioned within the chamber defined by shell 50 and to be coupled to shell 50 via manipulation mechanism 28 in the manner described elsewhere herein. In addition, when contoured seal element 44 is positioned in this manner, the contoured shape of contoured seal element 44, described in more detail below, allows for an effective seal to be maintained between compliant peripheral rim 46 and face 52 while at the same time allowing tubing portion 22c to extend from within the chamber defined by shell 50 to outside of the chamber defined by shell 50. Such an effective seal would not be possible if no contoured seal element 44 were to be used, as the circular shape of tubing portion 22c, if used alone, would lead to air gaps between compliant peripheral rim 46 and face 52 (and thus leak to leaks). The end of tubing portion 22c that extends out of mask 48 is structured to be coupled to a sample cell and associated sensor (not shown) to enable gas from within mask 48 (collected through nasal cannula 20) to be transported to the sample cell and sensor for measurement and/or analysis of constituents of and/or parameters relating to the gas.

Referring to FIG. 9, an isometric view of an exemplary embodiment of contoured seal element 44 is shown. Contoured seal element 44 is made of a soft or semi-rigid material, such as, without limitation, silicone, foam or TPE. Contoured seal element 44 has a geometry that includes flat base 54, contoured top surface 56 having a concave central portion 58 and end portions 60a and 60b that taper to meet flat base 54. Hole 62 is provided through the body of contoured seal element 44 for receiving and holding tubing portion 22c. It is this geometry of contoured seal element 44 that enables a good seal to be maintained between compliant peripheral rim 46 and face 52, thereby reducing the possibility leaks.

As still a further alternative, a fluid communicating device coupled to manipulation mechanism 28 could be provided wherein a part of a tube portion of the device itself could have the geometry of the contoured seal element 44 described above. That part of the tube portion would then be positioned between the flexible, compliant peripheral rim of a mask the patient's face in order to provide an effective seal, while manipulation mechanism 28 would allow the fluid communicating device to be selectively positioned from the exterior of the mask.

Thus, the present invention as described in connection with the various embodiments herein provides a solution that enables the position of an internal component of a patient interface device to be manipulated from the exterior of the patient interface device without the need to remove the patient interface device for the patient's and/or disturb a previously established seal between the patient interface device and the patient's face.

Although the invention has been described in detail for the purpose of illustration based on what is currently considered to be the most practical and exemplary embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A patient interface device, comprising:
a mask having a shell, wherein the shell includes an interior surface, an exterior surface, and defines a chamber;
a component structured to be selectively positioned within the chamber; and
a manipulation mechanism adapted to selectively manipulate a position of the component within the chamber from an exterior of the mask when the mask is placed against the face of the patient.

2. The patient interface device according to claim 1, wherein the manipulation mechanism includes:
a first attachment element attached to the component and structured to be selectively positioned on the interior surface of the shell; and
a first manipulation element structured to be selectively positioned on the exterior surface of the shell and operatively coupled to the first attachment element through the shell, wherein movement of the first manipulation element causes movement of the first attachment element and the component.

3. The patient interface device according to claim 2, wherein the first attachment element includes a first magnet, wherein the first manipulation element includes a second magnet, and wherein the first attachment element is coupled to the first manipulation element by a magnetic attraction between the first magnet and the second magnet through the shell.

4. The patient interface device according to claim 3, wherein the first manipulation element further includes a handle element coupled to the second magnet for selectively moving the manipulation element on the exterior surface of the shell.

5. The patient interface device according to claim 2, wherein the component comprises a nasal cannula coupled to a tubing portion.

6. The patient interface device according to claim 5, wherein the nasal cannula further comprises an interior port provided on the interior surface, and an exterior port provided on the exterior surface, wherein the interior port is in fluid communication with the exterior port, and wherein the at least one tubing portion is fluidly coupled to the interior port.

7. The patient interface device according to claim 2, wherein the manipulation mechanism includes:
a second attachment element attached to the component and structured to be selectively positioned on the interior surface of the shell; and
a second manipulation element structured to be selectively positioned on the exterior surface of the shell and coupled to the second attachment element through the shell, wherein movement of the second manipulation element causes movement of the second attachment element and the component.

8. The patient interface device according to claim 7, wherein the first attachment element includes a first magnet, wherein the first manipulation element includes a second magnet, wherein the first attachment element is coupled to the manipulation element by a first magnetic attraction between the first magnet and the second magnet through the shell, wherein the second attachment element includes a third magnet, wherein the second manipulation element includes a fourth magnet, and wherein the second attachment element is coupled to the second manipulation element by a second magnetic attraction between the third magnet and the fourth magnet through the shell.

9. The patient interface device according to claim 5, wherein the first attachment element includes a loop for receiving the tubing portion to attach the first attachment element to the tubing portion.

10. A method of adjusting a position of a component of a patient interface device having a mask having a shell having an interior surface and an exterior surface, comprising:
positioning an attachment element on the interior surface, the attachment element being attached to the component;
positioning a manipulation element on the exterior surface of the shell and coupling the manipulation element to the attachment element through the shell;
placing the mask against a face of a patient, the shell defining a chamber when the mask is placed against the face of the patient; and
moving the manipulation element on the exterior surface, wherein the movement of the manipulation element causes movement of the attachment element and movement of the component within the chamber.

11. The method according to claim 10, wherein the attachment element includes a first magnet, wherein the manipulation element includes a second magnet, and wherein the attachment element is coupled to the manipulation element by a magnetic attraction between the first magnet and the second magnet through the shell.

12. The method according to claim 10, wherein the component comprises a fluid communicating device, and wherein the method comprising collecting a gas from within the chamber using the fluid communicating device and communicating the gas out of the chamber while the mask is placed against the face of the patient.

13. The method according to claim 10, wherein communicating the gas out of the chamber comprises communicating the gas through the shell using the fluid communicating device.

14. A method of adjusting a position of a component of a patient interface device having a mask having a shell having an interior surface and an exterior surface, comprising:
positioning a first attachment element on the interior surface, the first attachment element being attached to the component;
positioning a first manipulation element on the exterior surface of the shell and coupling the first manipulation element to the first attachment element through the shell;
positioning a second attachment element on the interior surface, the second attachment element being attached to the component;
positioning a second manipulation element on the exterior surface of the shell and coupling the second manipulation element to the second attachment element through the shell;
placing the mask against a face of a patient, the shell defining a chamber when the mask is placed against the face of the patient; and
moving the first manipulation element on the exterior surface, wherein the movement of the first manipulation element causes movement of the first attachment element and movement of the component within the chamber, and moving the second manipulation element on the exterior surface, wherein the movement of the second manipulation element causes movement of the second attachment element and movement of the component within the chamber.

15. The method according to claim 14, wherein the first attachment element includes a first magnet, wherein the first manipulation element includes a second magnet, wherein the first attachment element is coupled to the first manipulation element by a first magnetic attraction between the first magnet and the second magnet through the shell, wherein the second attachment element includes a third magnet, wherein the second manipulation element includes a magnet, and wherein the second attachment element is coupled to the second manipulation element by a second magnetic attraction between the third magnet and the fourth magnet through the shell.

16. The method according to claim 14, wherein the component comprises a fluid communicating device, and wherein the method comprising collecting a gas from within the chamber using the fluid communicating device and communicating the gas out of the chamber while the mask is placed against the face of the patient.

17. The method according to claim 14, wherein the communicating the gas out of the chamber comprises communicating the gas through the shell using the fluid communicating device.

18. A fluid communicating device for a patient interface device having a mask having a shell, the shell having an interior surface and an exterior surface, the shell defining a chamber when the mask is placed against a face of a patient, comprising:
(a) a tubing portion; and
(b) a manipulation mechanism for selectively manipulating a position of the fluid communicating device within the chamber from an exterior of the mask when the mask is placed against the face of the patient, the manipulation mechanism comprising:
(1) an attachment element coupled to the tubing portion and structured to be selectively positioned on the interior surface of the shell, and
(2) a manipulation element structured to be selectively positioned on the exterior surface of the shell and coupled to the attachment element through the shell, wherein movement of the manipulation element when coupled to the attachment element causes movement of the attachment element and the fluid communicating device.

19. The fluid communicating device according to claim 18, wherein the attachment element includes a first magnet, wherein the manipulation element includes a second magnet, and wherein the attachment element is coupled to the manipulation element by a magnetic attraction between the first magnet and the second magnet through the shell.

20. The fluid communicating device according to claim 18, wherein the first magnet comprises two first disk shaped magnets and wherein the second magnet comprises two second disk shaped magnets.

21. The fluid communicating device according to claim 18, wherein the manipulation element includes a handle element coupled to the second magnet for selectively moving the manipulation element on the exterior surface of the shell.

22. The fluid communicating device according to claim 18, further comprising a nasal cannula coupled the tubing portion.

23. The fluid communicating device according to claim 18, wherein the manipulation mechanism includes:
a second attachment element coupled to the tubing portion and structured to be selectively positioned on the interior surface of the shell; and
a second manipulation element structured to be selectively positioned on the exterior surface of the shell and coupled to the second attachment element through the shell, wherein movement of the second manipulation element when coupled to the second attachment element causes movement of the second attachment element and the fluid communicating device.

24. The fluid communicating device according to claim 23, wherein the attachment element includes a first magnet, wherein the manipulation element includes a second magnet, wherein the attachment element is coupled to the manipulation element by a first magnetic attraction between the first magnet and the second magnet through the shell, wherein the second attachment element includes a third magnet, wherein the second manipulation element includes a fourth magnet, and wherein the second attachment element is coupled to the second manipulation element by a second magnetic attraction between the third magnet and the fourth magnet through the shell.
